(19) United States
(12) Patent Application Publication
(10) Pub. No.: US 2003/0171794 A1
(43) Pub. Date: Sep. 11, 2003

(54) Racket for providing electric pulse to human body in exercise

(76) Inventor: Arthur Hong, Taichung Hsien (TW)

Correspondence Address: BROWDY AND NEIMARK, P.L.L.C., 624 Ninth Street, N.W., Washington, DC, 20001-5303, US

(21) Appl. No.: 10/090,618

(22) Filed: Mar. 6, 2002

(51) Int. Cl. A61N 1/18

(52) U.S. Cl. 607/72

(57) ABSTRACT

A racket mounts an electric pulse generator thereon for providing electric pulses to human body when exercising. The electric pulse generator has a base board. A battery seat is disposed on the base board for installing at least one battery therein. A power controller is disposed on the base board for enlarging the potential and the current of the battery. A waver switch is disposed on the base board for switching-on and switching-off the power of the battery when the racket is waved, and two conducting members are disposed on a handle of the racket conducting to the positive and the negative pole terminals of the power controller. Whereby, user holds the handle of the racket to touch both of the conducting members to get electric pulses when waving the racket to switch the waver switch on.

### FIELD OF THE INVENTION

The present invention relates generally to a racket, and more particularly to a racket, which is capable of providing electric pulses to human body.

### BACKGROUND OF THE INVENTION

For the patients having musculoskeletal injure, doctor sometime will treat patients which electric stimulating. In Chinese medicine, doctors acupuncture patient with electric adding to the needle to cure the diseases. In modem life, more and more people accept electric treatment for health care. People stimulate muscles by electric pulses to relax muscle, help blood circulation or beauty treatment etc.

However, people only get electric treatment when rest. If people are in activity, such as when in exercise, there is no appropriate apparatus for people to get electric treatment when exercising, but not interfering with the activity.

### SUMMARY OF THE INVENTION

The primary object of the present invention is to provide a racket, which is capable of providing electric pulses to human body for user can get electric treatment when exercising.

According to the object of the present invention, an electric pulse generator is mounted on a racket for providing electric pulses to human body. The electric pulse generator has a base board. A battery seat is disposed on the base board for installing at least one battery therein. A power controller is disposed on the base board for enlarging the potential and the current of the battery. The power controller has a positive pole terminal and a negative pole terminal. A waver switch is disposed on the base board for switching-on and switching-off the power of the battery when the racket is waved. Two conducting members are disposed on a handle of the racket with one of which conducting to the positive pole terminal of the power controller, and the other one of which conducting to the negative pole terminal of the power controller. Whereby, user holds the handle of the racket to touch both of the conducting members to get electric pulses when the waver switch is switching on.

### BRIEF DESCRIPTION OF THE DRAWINGS

FIG. 1 is a front side view of a preferred embodiment of the present invention;

FIG. 2 is a perspective view of the preferred embodiment of the present invention, and

FIG. 3 is a schematic view of the preferred embodiment of the present invention, showing the racket being waved.

### DETAILED DESCRIPTION OF THE INVENTION

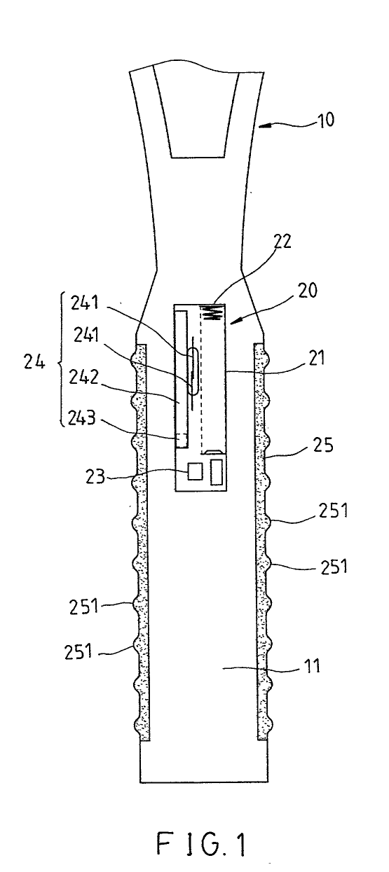

Please refer to FIG. 1, a preferred embodiment of the present invention provides a racket 10, which mounts an electric pulse generator 20 thereon. The electric pulse generator 20 comprises the following elements.

A base board 21 is disposed in a handle 11 of the racket 10 at topside thereof.

A battery seat 22 is disposed on the base board 21, which is to receive batteries (not shown) therein for providing the electric pulse generator 20 a required electric power.

A power controller 23, which is an IC chip in the present preferred embodiment, is disposed on the base board 21. The power controller 23 is to enlarge the potential and the current of the batteries. Such that, the output potential is in the range of 75 V to 125 V, and the output current is under 1 mA. If installing a 1.5 V battery into the battery seat 22, there are a potential about 100 V and a current about 0.3 mA to 0.5 mA outputting via the power controller 23.

A waver switch 24 is disposed on the seat board 21. The waver switch 24 has two conducting pieces 241 having parts thereof closed. An elongated guiding tube 242 is disposed beside the conducting pieces 241. The guiding tube 242 installs a magnetic member 243 therein. The magnetic member 243 can slide along the elongated axis of the guiding tube 242. While the magnetic member 243 approaches the closed potions of the conducting pieces 241, the magnetic force of the magnetic member 243 will attract one conducting piece 241 touching the other one conducting piece 241 to make the waver switch 24 switching the electric power on. On the contrary, while the magnetic member 243 slides away from the closed potions of the conducting pieces 241, the conducting pieces 241 will no longer touch to make the waver switch 24 switching the electric power off.

It is to be understood that the waver switch 24 is to switching-on and switching-off the electric power when the racket is waved. In the prior arts, there had various waver switches, such as working by mercury, would be the alternated of the present invention.

Two conducting members 25, which are piece-liked elements in the present preferred embodiment, have one of which conducting to the positive pole terminal (not shown) of the power controller 23, and the other one of which conducting to the negative pole terminal (not shown) of the power controller 23. The conducting members 25 are mounted at the surface of the handle 11 of the racket 10 respectively at opposite sides thereof. The conducting members 25 are made of rubber that capable of conducting electric. The conducting members 25 have protrusions 251, which are semi-ball elements in the present embodiment, at the exterior surfaces thereof respectively.

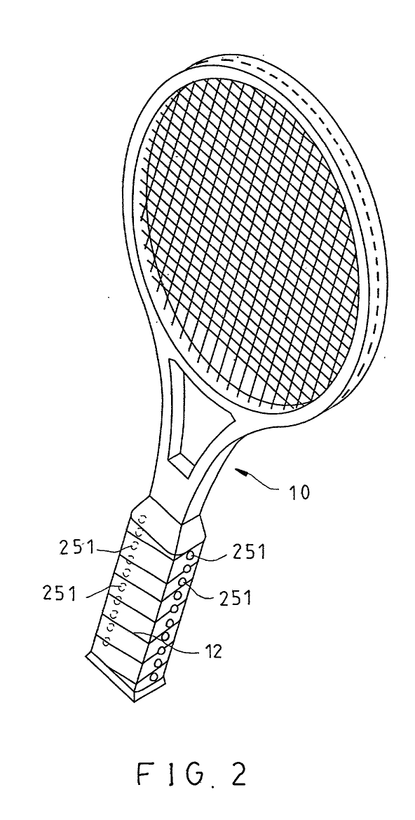

Please refer to FIG. 2, a holding strip 12 is winded on the handle 11 of the racket 10, which has openings thereon for the protrusions 251 of the conducting members 25 to be exposed. When a user holds the handle 11 of the racket 10 and touches both of the protrusions 251 for conducting electric pulses to human body.

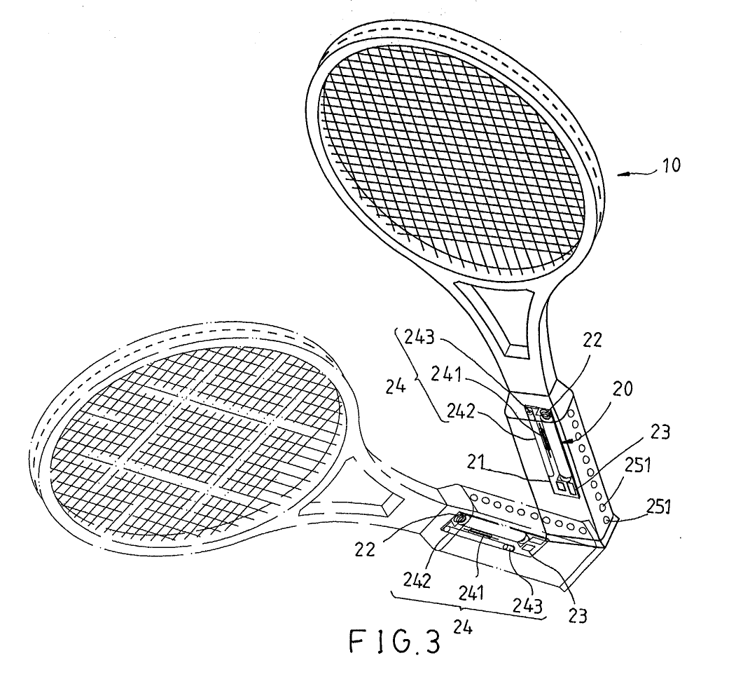

Please refer to FIG. 3, when a user holds the handle 11 and waves the racket 10, the hand will touch the protrusions 251 of both of the positive pole and the negative pole of the conducting members 25. An electric circuit will form between the hand and the conducting members 25. While the user waves the racket 10 to make the waver switch 24 suddenly switching-on and suddenly switching-off, the electric pulses will conduct to human body via the protrusions 251. The frequency of the electric pulses is according to the frequency of user waving the racket 10, which means the racket 10 of the present invention can provide user a variety of electric stimulating according to the exercise levels.

In conclusion, the racket 10 of the present invention can provide user electric pulses to massage muscles while exercising. The positions of the conducting potions can be arranged to correspond to the acupuncture points at hand to increase the effect of the electric stimulating. In addition, the frequency of the electric pulses provided by the racket 10 of the present invention is according to the times of user waving the racket 10. In a light exercise, the racket 10 of the present invention can provide user a lower electric stimulating. In a heavy exercise, the racket 10 of the present invention can provide user a larger electric stimulating.

A tennis racket is shown in FIGS. in the present invention. The electric pulse generator 20 also can be mounted on rackets for badminton, squash or racquetball etc., for players can get electric stimulating in the game.

What is claimed is:

1. A racket adapted for providing electric pulses to human body of the user, said racket comprising a handle and an electric pulse generator mounted on the handle, said electric pulse generator comprising:
a base board;
a battery seat disposed on said base board for installing at least one battery therein and adapted to provide the required electric power of the electric pulse generator;
a power controller disposed on said base board for enlarging the potential and the current of the battery, said power controller having a positive pole terminal and a negative pole terminal;
a waver switch disposed on said base board for switching-on and switching-off the power of the battery when the racket being waved, and
two conducting members disposed on the handle of the racket with one of which conducting to said positive pole terminal of said power controller, and the other one of which conducting to said negative pole terminal of said power controller, whereby the user holding the handle of the racket to touch both of said conducting members to get electric pulses when waving the racket to switch said waver switch on.

2. The racket as defined in claim 1, wherein said waver switch has two conducting pieces disposed closely with each other, a guiding tube beside said conducting pieces, and a magnetic member slidably disposed in said guiding tube such that said magnetic member approaches said switching pieces will attract one of said conducting pieces to touch the other so as to switch the battery on while the user is waving the racket.

3. The racket as defined in claim 1, wherein each of said conducting members has at least one protrusion thereon.

4. The racket as defined in claim 3 further comprising a holding strip winded on the handle and covered onto said conducting members, said holding strip having openings thereon corresponding in location to said protrusions said conducting members such that the protrusions can be exposed outside the holding strip.

5. The racket as defined in claim 3, wherein said conducting members are disposed at opposite sides of the handle respectively.
